(19) United States
(12) Patent Application Publication
(10) Pub. No.: US 2007/0260260 A1
(43) Pub. Date: Nov. 8, 2007

(54) SIZING INSTRUMENT FOR A BODILY JOINT SUCH AS AN INTERVERTEBRAL DISC SPACE

(76) Inventors: Michael R. Hahn, Oklahoma City, OK (US); Erik Emstad, St. Paul, MN (US); Steven Seme, Savage, MN (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/693,469

(22) Filed: Mar. 29, 2007

Related U.S. Application Data
(60) Provisional application No. 60/786,975, filed on Mar. 29, 2006.

(51) Int. Cl. A61B 17/60; A61F 5/00

(52) U.S. Cl. 606/102; 606/86

(57) ABSTRACT

A sizing instrument for measuring a bodily joint bounded by top and bottom surfaces, such as a disc space bounded by a top vertebra and a bottom vertebra, including a jaw assembly, a handle assembly, and an indicator portion. The jaw assembly includes an upper contact pad and a lower contact pad. The upper pad is adapted to substantially rigidly engage the top surface, and the lower pad is adapted to substantially rigidly engage the bottom surface. The handle assembly is linked to the jaw assembly and is adapted to vary a distance between the upper contact pad and the lower contact pad. In turn, the indicator portion is adapted to communicate the distance between the upper contact pad and the lower contact pad.

### CROSS-REFERENCE TO RELATED APPLICATIONS

The subject matter of this application is related to the subject matter of U.S. Provisional Application Ser. No. 60/786,975, filed Mar. 29, 2006 and entitled “Sizing Instrument for Bodily Joint Such as an Intervertebral Disc Space,” priority to which is claimed under 35 U.S.C. §119(e) and an entirety of which is incorporated herein by reference.

### BACKGROUND OF THE INVENTION

Various surgical procedures entail the need for estimating a size of an enclosed bodily joint, and typically require the use of one or more instruments. For example, prior to implanting a device into an intervertebral disc space/joint, a sizing instrument is normally employed to first estimate the size of the disc space so that an appropriately sized implant can be selected. Sizing instruments for measuring a size of a spinal disc space typically include a distal end that approximates a size and shape of an implant to be inserted into the disc space. Often times, the sizing instrument is impacted into the disc space with a mallet or other tool. A sequential sizing method (i.e., smallest to largest) is typically used, with the sizes of distal ends of the sizing instrument graduating until a desired fit is achieved. The desired fit is determined by a user based upon tactile feel, for example by determining whether the fit of the sizer in the disc space “feels” not too loose and not too tight. Based upon this subjective “feel test,” a final sizer is selected as an indicator of an appropriate implant size for a particular patient. Similar techniques are employed for estimating or measuring the size of other bodily joints.

Unfortunately, tactile feel is subjective and varies from person-to-person. Such directions as snug, or not too tight, while generally appropriate, leave room for some individual error. While surgeons have become adept at the sizing method described above, ensuring proper sizing techniques is important. For example, when the surgeon inadvertently selects a sizer instrument that is much larger than the space being evaluated, excessive impaction might be used to drive the distal end of the sizer instrument into the intervertebral disc space (or other bodily joint). As a result, the adjacent vertebrae could be over-distracted, resulting not only in damage to vertebral body endplates (or other bodily tissue or structure), but also damaging soft tissue stabilizers, which can result in an increase in iatrogenic instability, for example.

### SUMMARY OF THE INVENTION

Some aspects of the present disclosure relate to a sizing instrument for measuring a bodily joint having opposed, top and bottom surfaces, such as a disc space bounded by a top vertebra and a bottom vertebra. The sizing instrument includes a jaw assembly, a handle assembly, and an indicator portion. The jaw assembly includes an upper contact pad adapted to substantially rigidly engage the top surface, such as the top vertebra. The jaw assembly also includes a lower contact pad adapted to substantially rigidly engage the bottom surface, such as the bottom vertebra. The handle assembly is linked to the jaw assembly and is adapted to vary a distance between the upper contact pad and the lower contact pad. The indicator portion is associated with the handle assembly and is adapted to communicate the distance between the upper contact pad and the lower contact pad. The sizing instrument also optionally includes a biasing device in mechanical communication with the jaw assembly to bias the upper contact pad away from the lower contact pad, the biasing device adapted to provide a predetermined spring force for engaging the top and bottom surfaces of the bodily joint.

Other aspects of the present invention relate to a method of measuring a bodily joint of a human patient bounded by top and bottom surfaces, such as a disc space bounded by a top vertebra and a bottom vertebra. The method includes providing a sizing instrument including a jaw assembly, a handle assembly, and an indictor portion. The jaw assembly includes an upper contact pad and a lower contact pad. The handle assembly is in mechanical communication with the jaw assembly and is adapted to vary a distance between the upper contact pad and the lower contact pad. The indicator portion is adapted to communicate the distance between the upper contact pad and the lower contact pad. The method also includes collapsing the jaw assembly to bring the upper contact pad toward the lower contact pad such that the jaw assembly defines a minimized profile. The jaw assembly is inserted into the joint (e.g., disc space) and expanded against the top and bottom surfaces (e.g., top vertebra and the bottom vertebra) to substantially rigidly engage the surfaces. Following engagement, a height or other dimension of the bodily joint is obtained by a user via the indictor portion. The sizing instrument optionally includes a biasing device in mechanical communication with the jaw assembly to bias the upper contact pad away from the lower contact pad. A predetermined spring force of the biasing device is selected to reduce risk of over distraction of the bodily joint.

While some aspects of the invention have been described above, other related products and methods are also disclosed and provide additional advantages.

### BRIEF DESCRIPTION OF THE DRAWINGS

FIG. 1 is a side view of a sizing instrument, according to principles of the present disclosure;

FIG. 2 is a perspective view of a jaw assembly portion of the sizing instrument of FIG. 1 expanded to a first position;

FIG. 3 is a perspective view of the jaw assembly of FIG. 2 collapsed to a second position;

FIG. 4 is a perspective view illustrating a method of measuring a bodily joint, such as a disc space, according to principles of the present disclosure;

FIG. 5 is a simplified top view of a distal portion of an alternative sizing instrument; and

FIG. 6 illustrates use of the instrument of FIG. 5 in obtaining dimensional information of a bodily joint.

### DETAILED DESCRIPTION OF THE INVENTION

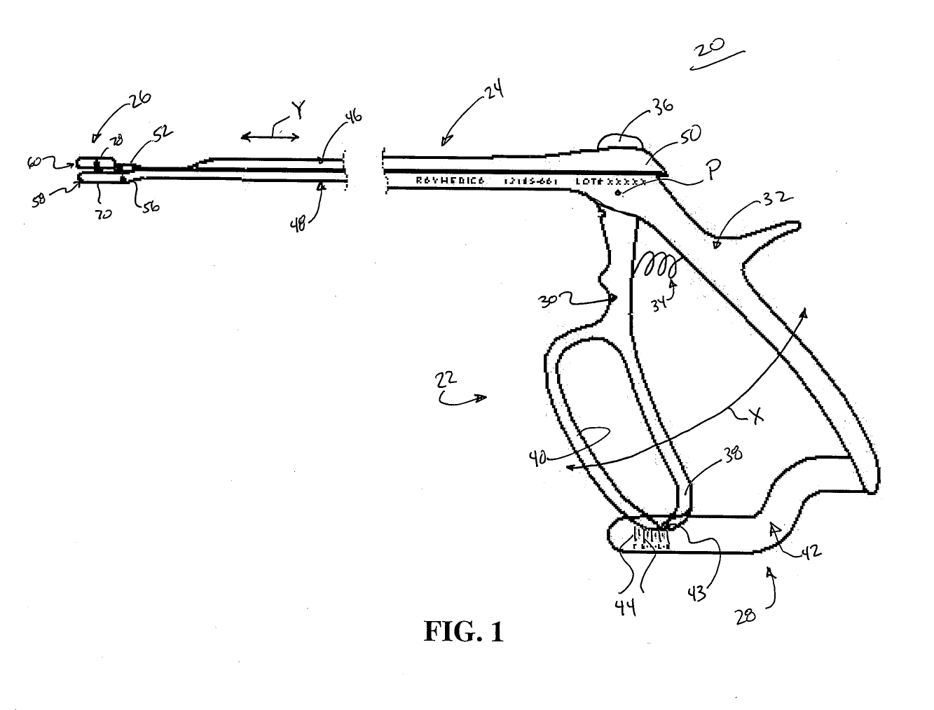

FIG. 1 illustrates one embodiment of a sizing instrument 20 in accordance with principles of the present disclosure. The sizing instrument 20 includes a handle assembly 22, an extension assembly 24, and a jaw assembly 26. Details on the various components are provided below. In general terms, however, the extension assembly 24 extends between the handle assembly 22 and the jaw assembly 26, and provides mechanical communication between the assemblies 22, 26. The handle assembly 22 has an indicator portion 28 and is adapted to expand and collapse the jaw assembly 26 when actuated by a user. In some embodiments, the handle assembly 22 is configured to bias the jaw assembly 26 toward an expanded state. In turn, the indicator portion 28 is optionally adapted to provide information as to an overall separation distance (e.g., height) of the jaw assembly 26. The sizing instrument 20, and its component parts, can be formed of surgically safe materials, including polymeric and/or metallic materials, that can be sterilized and re-used.

In some embodiments, the handle assembly 22 includes a lever arm 30, a grip arm 32, a biasing device 34, and the indicator portion 28. The lever arm 30 extends from a first end 36 to a second end 38. The lever arm 30 is adapted to be grasped by a user, for example incorporating ergonomic features or other features facilitating a comfortable secure hand hold on the lever arm 30, such as a loop 40 sized to receive a user's fingers. The grip arm 32 is also optionally substantially ergonomically shaped or otherwise adapted to facilitate grasping, for example with a palm of the user's hand. While the handle assembly 22 is shown as incorporating a looped, pistol-style configuration, a wide variety of other configurations are also acceptable, adapted to facilitate grasping/actuation by a user's hand.

The lever and grip arms 30, 32 are pivotably assembled to one another at a pivot point P. With this in mind, the biasing device 34 is configured to bias the lever arm 30 (and in particular the second end 38) away from (or alternatively toward) the grip arm 32, with the arms 30, 32 pivoting relative to one another at the pivot point P. The biasing device 34 is characterized by a spring force or a spring constant, and in some embodiments is a spring. In one embodiment, the spring force is pre-selected to bias the second end 38 of the lever arm 30 arm away from the grip arm 32 at a predetermined force. As described below, the handle assembly 22, and thus the biasing device 34, is mechanically linked to the jaw assembly 26 such that the spring force associated with the biasing device 34 is transferred to the jaw assembly 26. As such, the biasing device 34 can assume a variety of other forms and can be assembled as other locations along the instrument 20 (including locations other than the handle assembly 22) so long as a biasing force is applied to the jaw assembly 26.

In some embodiments, the indicator portion 28 of the handle assembly 22 includes a scale member 42 and a pointer 43. The scale member 42 can extend from the grip arm 32 and includes a plurality of graduated markings 44 or other indicia adapted to communicate a dimension, such as the overall separation distance (e.g., height) of the jaw assembly 26 as described below. The pointer 43 is formed by, or extends from, the second end 38 of the lever arm 30, and is configured to highlight an individual one of the graduated markings 44 when positioned in close proximity thereto. For example, the pointer 43 can be a gap defined in the second end 38. Regardless, the lever arm 30 is associated with the scale member 42 such that the second end 38 (or the pointer 43) travels over the graduated markings 44 as the lever arm 30 is actuated relative to the grip arm 32. To this end, a position of the pointer 43 relative to individual ones of the graduated markings 44 is correlated with a position of the jaw assembly 26 as described below.

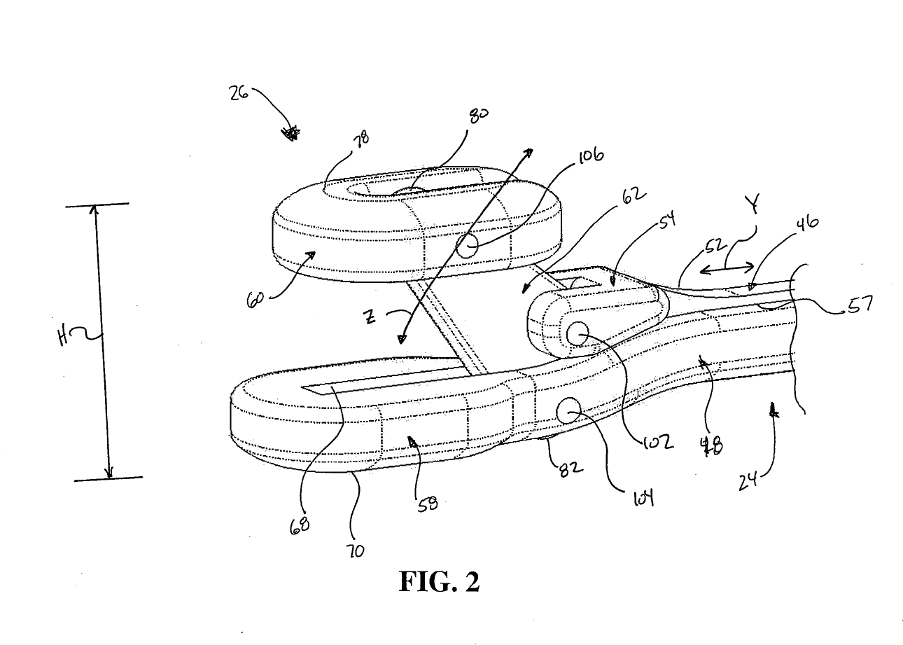

The extension assembly 24 defines a length convenient for inserting the jaw assembly 26 into a bodily joint, for example a disc space, by a user otherwise grasping the instrument 20 at the lever and grip arms 30, 32. The extension assembly 24 includes, in some embodiments, a slider arm 46 and a base 48. The slider arm 46 is a substantially elongate body extending from a proximal end 50 to a distal end 52. The proximal end 50 is connected to the lever arm 30 such that the slider arm 30 moves with the rotation of the lever arm 30 about the pivot point P. Further, the slider arm 46 forms or includes a yoke 54 (best shown in FIG. 2) at the distal end 52. The base 48 extends from the grip arm 32 to a distal end 56, and in some embodiments is integrally or homogenously formed with the grip arm 32.

As reflected in FIG. 1, upon final assembly, the slider arm 46 and the base 48 extend in a generally parallel fashion from the handle assembly 22, with the slider arm 46 being slidable relative to the base 48. To facilitate this relationship, in some embodiments, the base 48 forms a slot 57 (referenced generally in FIG. 2) within which a portion of the slider arm 48 is slidably received. Alternatively, one or both of the slider arm 46 and/or base 48 can include or form other features that promote movable assembly therebetween.

With reference to FIG. 2, the jaw assembly 26 includes a lower contact pad 58, an upper contact pad 60, and a connector 62, in some embodiments. As described below, the connector 62 links the pads 58, 60 relative to one another, as well as to the extension assembly 24.

The lower contact pad 58 can have a height on the order of 1-10 mm, for example 3 mm, and a width on the order of 3-15 mm, for example 8 mm, although other dimensions are contemplated. The lower contact pad 58 defines a substantially oval-shaped profile, forms a slot 68 sized to slidably receive the connector 62, and defines a lower contact surface 70. The lower contact pad 58 is adapted to forcibly, or otherwise substantially rigidly engage or contact bodily tissue during a sizing procedure (e.g., an endplate of a vertebra) with the lower contact surface 70. In this regard, the lower contact surface 70 is smooth and free of comers so as to minimize traumatic interaction with tissue. Further, a leading end 72 is curved and/or smooth, thus presenting an atraumatic surface for initial insertion into a bodily joint (or other bodily structure being measured). Additionally, the lower contact pad 58 is optionally substantially continuously formed with the base 48 of the extension assembly 24, extending distally from the distal end 56 of the base 48.

The upper contact pad 60 is optionally substantially U-shaped, defining a slot 76 with rounded edges and an upper contact surface 78. The upper contact pad 60 can have a height on the order of 1-10 mm, for example 3 mm, and a width on the order of 3-15 mm, for example 8 mm, although other dimensions are contemplated. As will be described subsequently in greater detail, the upper contact pad 60 is adapted to forcibly, or otherwise substantially rigidly, engage or otherwise contact bodily tissue during a sizing procedure (e.g., an endplate of a vertebra or other bodily joint surface) with the upper contact surface 78. Thus, the upper contact surface 78 is, similar to the lower contact surface 70, smooth and free of corners. Further, a leading end 79 of the upper contact pad 60 is curved and/or smooth for atraumatic insertion into a bodily joint.

In some embodiments, the connector 62 is generally triangular in shape defining a first corner 80 (shown partially obscured), a second corner 82 (shown partially obscured), and a third corner (hidden within the yoke 54). With the one configuration of FIG. 2, the connector 62 has a thickness commensurate with the slots 68, 76 so as to be slidably received therein. Alternatively, the connector 62 can assume a variety of other shapes appropriate for effectuating the linkage described below. Even further, the connector 62 can include two or more discrete bodies connected to one another.

With reference to FIG. 1, assembly of the sizing instrument 20 can be described as follows. The lever arm 30 is rotatably secured to the grip arm 32 at the pivot P proximate the first end 36 of the lever arm 30. The biasing device 34 is secured to the lever arm 30 and the grip arm 32 adjacent (e.g., below) the pivot P, and exerts a force between the lever arm 30 and the grip arm 32, for example to bias the second end 38 of the lever arm 30 away from the grip arm 32.

The scale member 42 is secured to (or integrally formed with) the grip arm 32 and extends proximate the second end 38 of the lever arm 30. In particular, the scale member 42 extends such that the pointer 43 (in association with the second end 38 of the lever arm 30) is selectively aligned relative to respective ones of the graduated markings 44.

The slider arm 46 extends at least partially within the slot 57 (FIG. 2) of the base 48, and is slidable distally and proximally relative thereto. The slider arm 46, and in particular the proximal end 50 of the slider arm 46, is secured relative to the lever arm 30 in such a manner that actuation of the lever arm 30 toward and away from the grip arm 32 (pivoting about the pivot point P), as designated generally by the curved line X in FIG. 1, results in distal and proximal motion, respectively, of the slider arm 46 relative to the base 48, as designated generally by the line Y.

With reference to FIG. 2, the yoke 54 of the slider arm 46 is rotatably secured to the third corner of the connector 62 at a pivot 102 (e.g., a pin), in one embodiment. In turn, the second corner 82 of the connector 62 is rotatably secured to the distal end 56 of the base 48 at a pivot 104 (e.g., a pin). The upper contact pad 60 is rotatably secured to the first corner 80 of the connector 62 at a pivot 106 (e.g., a pin). In this manner, the upper contact pad 60, the slider arm 46, the distal end 56 of the base 48, and the connector 62 form a linkage such that moving the slider arm 46 proximally and distally relative to the base 48 (as designated by the line Y) results in movement of the upper contact pad 60 toward and away from the lower contact pad 58 as designed generally by the line Z. In particular, the upper contact surface 78 and the lower contact surface 70 define an overall height or separation distance H of the jaw assembly 26 which can be varied according to the linkage principle described, for example.

With additional reference to FIG. 1, because the slider arm 46 is connected to the lever arm 30, it will be understood that the handle assembly 22 is in mechanical communication with the jaw assembly 26 via the extension assembly 24. In particular, actuation of the lever arm 30 relative to the grip arm 32 results in actuation of the upper contact pad 60 toward and away from the lower contact pad 58. Furthermore, as the lever arm 30 and the grip arm 32 are secured to the biasing device 34, it will also be understood that the biasing device 34 is in mechanical communication with jaw assembly 26 as it biases the lever arm 30 away from the grip arm 32. In this manner, the jaw assembly 26 is biased toward expansion of the lower and upper contact pads 58, 60 away from one another, but can be collapsed upon squeezing the lever arm 30 toward the grip arm 32, as well as allowed to expand by releasing the lever arm 30 (such that a force of the biasing device 34 naturally forces the lever arm 30 away from the grip arm 32), to define varying overall heights H between the upper contact surface 78 of the upper contact pad 60 and the lower contact surface 70 of the lower contact pad 58. A desired spring force of the biasing device 34 is optionally selected in order to pre-select a force at which the lower and upper contact pads 58, 60 are biased away from one another. As will be described subsequently, the spring force is optionally selected to help reduce risk of over distraction of opposed vertebrae, or structures of other bodily joints, during a sizing procedure.

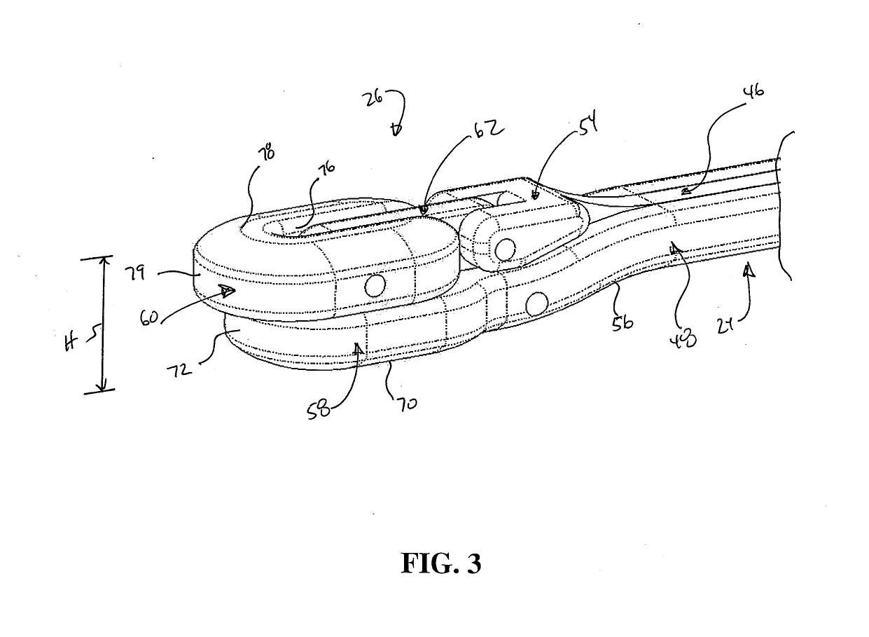

With reference to FIG. 3, the jaw assembly 26 is shown in a fully collapsed state, with the upper contact pad 60 collapsed fully toward the lower contact pad 58. In the fully collapsed state, the jaw assembly 26 defines a minimized overall height profile. For example, the yoke 54 slides distally from the position shown in FIG. 2 along the base 48 and slightly downwardly following a curve in the base 48 at the distal end 56. In this manner, the yoke 54 does not protrude or otherwise project above the upper contact surface 78 of the upper contact pad 60. Additionally, the connector 62 is in a substantially prone position, and does not protrude or otherwise project from the slot 76 of the upper contact pad 60. Similarly, the connector 62 is also disposed within the slot 68 (FIG. 2) of the lower contact pad 58 and does not protrude or otherwise project below the lower contact surface 70. Thus, the contact pads 58, 60 combine to define a maximum height of the jaw assembly 26 that is not less than a combined height of the yoke 54 and the base 48, or of the extension assembly 24 immediately proximal the jaw assembly 26.

With the above construction, the jaw assembly 26 can define an overall minimized profile in the collapsed state or position for optimal insertion into a bodily joint, such as a disc space. In some embodiments, the minimized overall profile is substantially smaller than an implant (not shown) to be inserted into the bodily joint (e.g., disc space) following measurement. As such, insertion of the jaw assembly 26 into the bodily joint to be measured does not require a larger hole than that required for implant insertion.

With the above in mind, a method of adjusting the overall spacing or height H defined by the contact pads 58, 60 is described as follows. In the absence of an external user-supplied squeezing force, the biasing device 34 biases the second end 38 of the lever arm 30 away from the grip arm 32 (i.e., clockwise relative to the orientation of FIG. 1) at a predetermined force such that the jaw assembly 26 is biased to an open or expanded state, with the upper contact pad 60 away from the lower contact pad 58 (i.e., the state or position of FIG. 2). The user then grasps and squeezes the lever arm 30 and the grip arm 32 to effectuate collapse of the jaw assembly 26 as described above (via operation of the extension assembly 24 and the connector 62), for example to the fully collapsed state or position shown in FIG. 3. The user then releases one or both of the lever arm 30 and/or the grip arm 32 (or otherwise reduces the applied squeezing force placed upon the arms 30, 32) to allow the jaw assembly 26 to self-expand at the predetermined force (i.e., the force constant of the biasing device 34). During expansion and collapsing of the jaw assembly 26, the second end 38/pointer 43 of the lever arm 30 continuously points to the graduated markings 44 on the scale member 42 which correspond to a numerical value for the overall height H.

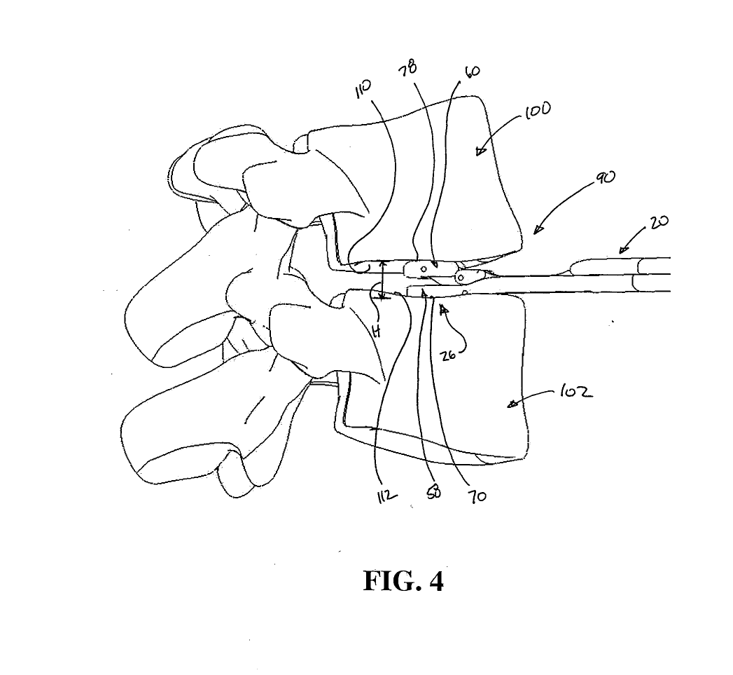

With reference to FIG. 4, a method of measuring a size or dimension of a bodily joint 90 in accordance with principles of the present disclosure is described. With the one embodiment of FIG. 4, the bodily joint 90 being measured is an intervertebral disc space, it being understood that a number of other bodily joints outside of the spine can also be measured (e.g., hip joint, knee joint, etc.) in accordance with the present disclosure. With this in mind, and by way of background, the disc space 90 is defined by a surrounding annulus fibrosis (not shown), a top vertebra 100, and a bottom vertebra 102 opposing the top vertebra 100, and contains a nucleus material (not shown). Each of the top and bottom vertebrae 100, 102 defines an endplate 110, 112, respectively. The endplates 110, 112 generally define the top and bottom, or overall height, of the disc space 90.

The method includes first accessing the disc space 90. In one embodiment, an anterior retro-peritoneal approach (ARPA) is used. An annular opening is created, forming a window in the annulus fibrosis (not shown) capable of receiving (or allowing passage of) the jaw assembly 26. Where the ARPA technique is used, a more centrally located anterior incision or window is optionally formed. In turn, where a posterior approach to the intervertebral disc 90 is used, the incision or window in the annulus fibrosis is at an offset from a posterior-anterior centerline of the disc space 90. A desired amount of the disc nucleus (not shown) is optionally removed, for example substantially the entire disc nucleus.

With additional reference to FIG. 1, the user initiates the sizing procedure by grasping the handle assembly 22 and applying a squeezing-type force to the lever and grip arms 30, 32. When a force sufficient to overcome a force of the biasing device 34 is applied (i.e., the lever arm 30 rotates relative to the grip arm 32 about the pivot P), the jaw assembly 26 is caused to transition from the expanded state or position (FIG. 2) to the collapsed state or position (FIG. 3), and thus to the minimized height H or profile (FIG. 2). The jaw assembly 26 is then inserted into the disc space 90 through the window in the annulus fibrosis. Where the ARPA technique is used, a more central approach that is not substantially angularly offset in vertical or lateral directions is made into the disc space 90, such that the jaw assembly 26 is able to be inserted into the disc space 90 at a more centrally located position that is substantially along the posterior-anterior centerline of the disc space 90. In other embodiments, where a posterior technique is used, an offset approach (angularly in a vertical direction and laterally toward an annular margin of the intervertebral disc) is made into the disc space 90 such that the jaw assembly 26 is inserted at an offset to the posterior-anterior centerline.

Once the contacts pads 58, 60 are within the joint to be measured, the user then removes the squeezing force being applied to the handle assembly 22 (e.g., the user releases the lever arm 30 while still holding the grip arm 32). As referenced above, the biasing device 34 is in mechanical communication with the jaw assembly 26 via the extension assembly 24. In this manner, the biasing device 34 self-biases the lower and upper contact pads 58, 60 away from one another at a predetermined force until the pads 58, 60 contact or engage the top and bottom vertebrae 100, 102, respectively. In one embodiment, the predetermined force is selected to avoid overt distraction and/or trauma of the bodily joint being sized. For example, for intervertebral disc space sizing, the force constant of the biasing device 34 is selected such that a maximum expansion force exerted by the pads 58, 60 upon the vertebrae 100, 102 will not exceed 50N. For other applications (e.g., sizing of bodily joints other than a disc space), a greater (i.e., greater than 50 N) or lesser or maximum expansion force can be selected. In fact, in some embodiments, the predetermined force can be selected or adjusted to match the constraints or needs of a subsequently implanted input device. Regardless, the jaw assembly 26 expands until the top and bottom vertebrae 100, 102 are substantially rigidly engaged, with a distance between the upper and lower contact surfaces 70, 78 reflecting a height (or other dimension) of the bodily joint (e.g., height of the disc space 90). This measured dimension is indicated to the user at indicator portion 28. In particular, the user is able to take a reading for the joint dimension in question by reading the location of the second end 38/pointer 43 of the lever arm 30 on the graduated markings 44, for example. In this manner, the user is able to obtain a height measurement of the disc space 90 (or other joint dimension) at a controlled and predetermined force selected to avoid over distraction of the top and bottom vertebrae 100, 102. Where desired, the jaw assembly 26 can be partially retracted and maneuvered to other locations within the joint space and additional measurements obtained. The handle assembly 22 is then squeezed to fully collapse the jaw assembly 26, and the jaw assembly 26 is then retracted from the disc space 90.

According to the techniques described above, a center of the disc space 90 is optionally measured, for example by using the ARPA technique to have a more centrally located approach at the disc space 90 during insertion of the sizing instrument 20, rather than a measurement taken at an angular offset or a measurement taken at the annular margin. In one embodiment, by measuring the center of the disc space 90, the overall height H between the top and bottom contact surfaces 70, 78 is taken at a location of a properly positioned spinal implant (not shown). Where an approach is made into the disc space 90 at an angular offset in the vertical direction and/or at a lateral offset from the posterior-anterior centerline toward the annular margin, the overall height measured may not be as indicative of a height of the disc space 90 where an implant will be placed. For example, if the end plates 110, 112 are substantially concave then an overall height of the disc space 90 at the annular margin is potentially less than an overall height of the disc space 90 at a location in the disc space 90 where the implant is properly positioned. In sum, less desirable readings may be taken where an angle of approach is more vertical and/or laterally offset toward the annular margin. In at least this manner, the ARPA technique is particularly advantageous in some applications.

In one embodiment, the instrument 20 is used in connection with a procedure for implanting a spinal implant (not shown) into the disc space, it being understood that the instrument 20 and method in accordance with aspects of the present invention is equally applicable to other spine-related procedures (e.g., fusion and non-fusion surgical procedures) as well as procedures related to bodily joints apart from the spine. With this one embodiment, however, the jaw assembly 26 is adapted to be substantially smaller than a size and shape of the spinal implant (not shown) being inserted into the disc space 90. In particular, the lower and upper contact pads 58, 60 taken together are substantially smaller in size and shape (e.g., height and width) in comparison to the implant when the jaw assembly 26 is in the minimized overall profile state. However, various dimensions are contemplated, including the lower and upper contact pads 58, 60 being about the same size as, or larger than, the implant to be inserted. The spinal implant is optionally a prosthetic spinal disc nucleus (not shown), such as those available from Raymedica, LLC of Bloomington, Minn. The prosthetic disc nucleus includes an outer jacket (not shown) surrounding an expandable core (not shown) formed of a hydrogel material, which upon hydration, expands to, and is constrained by, the outer jacket. Exemplary hydrogel core implants in accordance with the present invention are described in U.S. Pat. Nos. 5,824,093 and 6,132,465, the teachings of which are incorporated herein by reference.

It should also be understood that the sizing instrument 20, and in particular the jaw assembly 26, is optionally adapted for sizing the disc space 90 for other spinal implants, including other types of hydrogel core implants or implants using springs or disc replacement devices or other mechanical means of supporting the disc space 90. Thus, the sizing instrument 20 is in no way limited to any one particular spinal implant configuration (can be used with fusion or non-fusion procedures), nor is it limited to use with disc space applications.

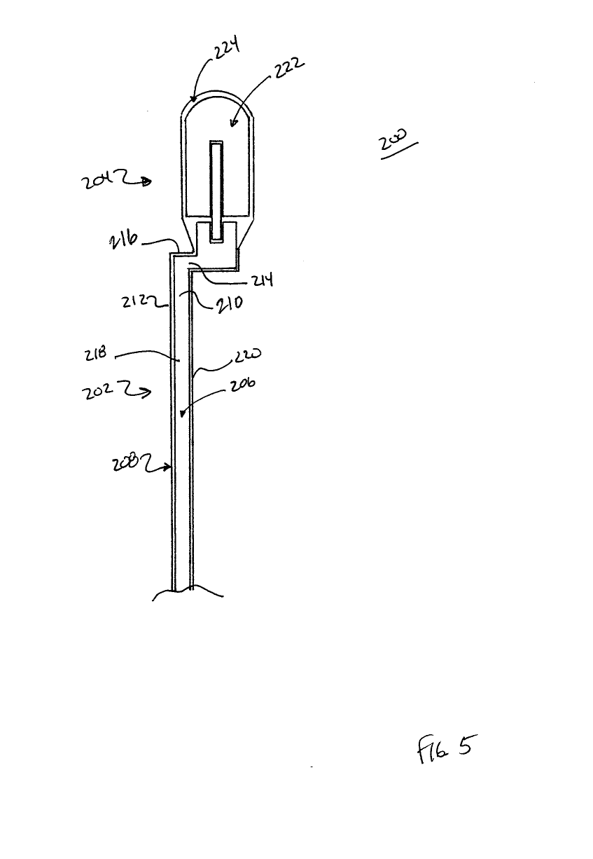

FIG. 5 illustrates a distal portion of an alternative sizing instrument 200 in accordance with principles of the present disclosure. The instrument 200 includes the handle assembly (not shown, but akin to the handle assembly 22 of FIG. 1), an extension assembly 202, and a jaw assembly 204. The jaw assembly 204 can assume a variety of forms, and in some embodiments is akin to the jaw assembly 26 (FIG. 2) previously described. Regardless, the extension assembly 202 links the handle assembly and the jaw assembly 204 to facilitate user-dictated transitioning of the jaw assembly 204 between a collapsed state and an expanded state, as with previous embodiments. With the configuration of FIG. 5, however, the extension assembly 202 includes or provides a transverse offset position of the jaw assembly 202 as described below.

More particularly, the extension assembly 202 includes a slider arm 206 and a base 208. The slider arm 206 and the base 208 are connected at proximal ends (not shown) thereof to the handle assembly (not shown). Further, the slider arm 206 is slidably retained by the base 208 such that a distal segment 210 of the slider arm 206 is axially moveable relative to a distal segment 212 of the base 208. In this regard, each of the distal segments 210, 212 includes a shoulder 214, 216 that extends transversely relative to or from a corresponding intermediate segment 218, 220 respectively. With this arrangement, then, the distal segments 210, 212 locate the jaw assembly 204 so as to be offset from a centerline or axis of the intermediate segments 218, 220.

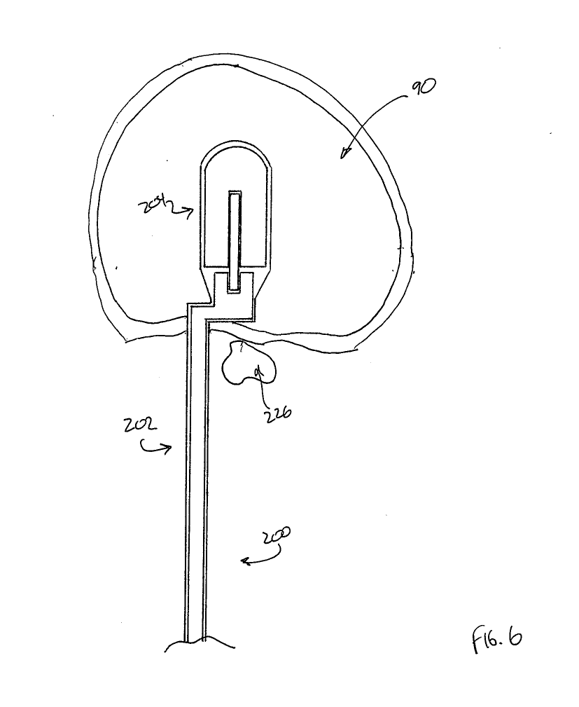

During use, the instrument 200 is employed to perform a bodily joint sizing procedure as previously described. In this regard, the offset arrangement of the jaw assembly 204 relative to the extension assembly 202 (and in particular the intermediate segments 214, 216) can facilitate desired positioning of the jaw assembly 204, including upper and lower contact pads 222, 224, while avoiding various anatomical structures of concern. For example, as shown in FIG. 6, for spinal disc space sizing applications, the jaw assembly 204 can be posteriorly inserted into the disc space 90, with the extension assembly 202 avoiding primary nerve structures 226.

In the foregoing Detailed Description, reference is made to the accompanying drawings which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The foregoing detailed description, therefore, is not to be taken in a limiting sense, and the scope of the present invention is defined by the appended claims.

What is claimed is:

1. A sizing instrument for measuring a bodily joint bounded by top and bottom surfaces, such as a disc space bounded by a top vertebra and a bottom vertebra, the sizing instrument comprising:
a jaw assembly comprising:
an upper contact pad adapted to substantially rigidly engage the top surface,
a lower contact pad adapted to substantially rigidly engage the bottom surface,
wherein the upper contact pad and the lower contact pad are movably linked;
a handle assembly linked to the jaw assembly and adapted to be actuated to vary a distance between the upper contact pad and the lower contact pad; and
an indicator portion associated with the handle assembly and adapted to communicate the distance between the upper contact pad and the lower contact pad.

2. The sizing instrument of claim 1, further comprising:
a biasing device in mechanical communication with the jaw assembly to bias the upper contact pad away from the lower contact pad.

3. The sizing instrument of claim 1, further comprising:
an extension assembly connecting the handle assembly and the jaw assembly.

4. The sizing instrument of claim 3, wherein a major axis of the extension assembly is non-concentric with the jaw assembly.

5. The sizing instrument of claim 3, wherein the handle assembly includes a lever arm pivotably connected to a grip arm, and further wherein the extension assembly comprises:
a slider arm connected to, and extending distally from, the lever arm; and
a base connected to, and extending distally from, the grip arm;
wherein the slider arm is slidably associated with the base.

6. The sizing instrument of claim 5, wherein a distal end of the slider arm forms a yoke.

7. The sizing instrument of claim 6, wherein the jaw assembly includes a connector linked to the upper and lower contact pads, and further wherein the yoke is pivotably mounted to the connector.

8. The sizing instrument of claim 6, wherein the base forms a slot at a distal region thereof, and further wherein the slider arm is received in the slot.

9. The sizing instrument of claim 8, wherein the yoke is slidably positioned along a surface of the base.

10. The sizing instrument of claim 9, wherein a combined height of the yoke and the base is not greater than a combined height of the contact pads.

11. The sizing instrument of claim 1, wherein the contact pads each define a curved leading end.

12. The sizing instrument of claim 1, wherein the contact pads each have a height on the order of 1-10 mm.

13. The sizing instrument of claim 1, wherein the indicator portion is integrally formed with the handle assembly.

14. The sizing instrument of claim 13, wherein the indicator portion includes indicia and a pointer corresponding with a distance between opposing surfaces of the contact pads.

15. A method of measuring a bodily joint of a patient bounded by top and bottom surfaces, such as a disc space bounded by a top vertebra and a bottom vertebra, the method comprising:
providing a sizing instrument comprising:
a jaw assembly comprising:
an upper contact pad adapted to contact the top surface,
a lower contact pad adapted to contact the bottom surface,
a handle assembly adapted to be actuated to vary a distance between the upper contact pad and the lower contact pad,
an indicator portion adapted to communicate the distance between the upper contact pad and the lower contact pad;
collapsing the jaw assembly to bring the upper contact pad toward the lower contact pad such that the jaw assembly defines a minimized profile;
inserting the collapsed jaw assembly into the bodily joint;
expanding the jaw assembly such that the upper contact pad engages the top surface and the lower contact pad engages the bottom surface; and
obtaining a dimensional measurement of the bodily joint with the indicator portion.

16. The method of claim 15, wherein the sizing instrument further comprises a biasing device in mechanical communication with the jaw assembly to bias the upper contact pad away from the lower contact pad, the method further comprising:
selecting a predetermined spring force of the biasing device.

17. The method of claim 16, wherein the predetermined spring force is selected based upon a factor selected from the group consisting of:
distraction limitations of the bodily joint, trauma limitations of the bodily joint and constraints of a subsequently implanted implant device

18. The method of claim 15, wherein the method is performed in evaluating a size of a spinal disc space bounded by an annulus and opposing top and bottom vertebrae, the method further comprising:
forming an opening in the annulus;
inserting the jaw assembly in the minimized profile through the opening and into the disc space; and
causing the jaw assembly to expand.

19. The method of claim 18, wherein causing the jaw assembly to expand includes:
self-biasing the upper and lower contact pads away from one another such that the upper contact pad engages the top vertebrae and the lower contact pad engages the lower vertebrae.

20. The method of claim 19, further comprising:
repeatedly collapsing and expanding the jaw assembly within the disc space to obtain multiple dimensional measurements of the disc space.
